(19) United States
(12) Patent Application Publication
(10) Pub. No.: US 2025/0065003 A1
(43) Pub. Date: Feb. 27, 2025

(54) A DEVICE FOR RETAINING AN OBJECT IN A STERILIZATION/DISINFECTION APPARATUS

(71) Applicant: Saban Ventures Pty Limited, Macquarie Park, NSW (AU)

(72) Inventors: Timothy Edward MONTANARO, Macquarie Park (AU); Rigor Del Mundo ASPA, Sutherland, NSW (AU); Joseph Samuel ORMORD, Mount Colah, NSW (AU); Gianluigi BORTOLUZZI, Macquarie Park (AU); Christopher HIBBARD, North Balgowlah, NSW (AU); Steven Paul FARRUGIA, Macquarie Park (AU)

(21) Appl. No.: 18/721,425

(22) PCT Filed: Dec. 16, 2022
(86) PCT No.: PCT/AU2022/051521

§ 371 Date: Jun. 18, 2024

(30) Foreign Application Priority Data
Dec. 22, 2021 (AU) 2021904207

(51) Int. Cl. A61L 2/26

(52) U.S. Cl. CPC A61L 2/26; A61L 2202/122

(57) ABSTRACT

A device for retaining a medical apparatus in a sterilisation or disinfection apparatus, said device comprising a closed cradle configured to fully encircle a portion of said medical apparatus when in use, the cradle having a medical apparatus-facing side comprising a plurality of projections for contact with the medical apparatus, and a cradle support configured to interface with the sterilisation or disinfection apparatus and retain the cradle within the sterilisation or disinfection apparatus during the operation of the sterilisation or disinfection apparatus.

### FIELD OF INVENTION

The present invention relates to a device for retaining an object in a sterilisation apparatus. More particularly, the present invention relates to devices for retaining a medical apparatus, such as an ultrasound probe, in a sterilisation apparatus. For convenience, much of this application will reference that context; however, it will be appreciated that the invention is not limited to this particular field of use.

### BACKGROUND

The following discussion of the prior art is intended to place the invention in an appropriate technical context and enable the associated advantages to be fully understood. However, any discussion of the prior art throughout the specification should not be considered as an admission that such art is widely known or forms part of the common general knowledge in the field.

Ultrasound probes are used for a variety of intra cavity procedures including intra rectal, intra vaginal and oesophageal examination as well as surface use. These probes are typically constructed integrally with the power and data cord used to link the probe with a control console.

The probes are relatively expensive and require disinfection or sterilization between patient use to avoid cross-contamination. It should be appreciated that the terms “disinfection” and “sterilization” are used interchangeably throughout this application as the described concepts can be applicable in either context. Similarly, that the terms “disinfect” and “sterilize” are used interchangeably throughout this application.

Probes can be cleaned manually after use but for high throughput and reliably reproducible certifiable disinfection, as well as operator safety, an automated disinfection or sterilisation process is preferred. Automated disinfection or sterilisation apparatus typically carry out the disinfection or sterilisation of the probe within a hollow chamber. When such an apparatus is used, the probe is typically retained in position inside the chamber by using a resilient clamp to engage the probe's cord, thereby allowing the probe to be suspended in position within the cavity. The surface of the probe is not in contact with any other object or surface. Once the probe suitably positioned, the chamber of the disinfection or sterilisation apparatus is sealed.

The disinfection or sterilization process then begins, in which a disinfectant or sterilant gas, spray or aerosol, usually a peroxy compound, is applied to the exposed surfaces of the probe for a predetermined time. After this phase is complete, the probe and the chamber are evacuated and dried if necessary. The chamber is then unsealed and the probe is removed ready for use.

In an alternative approach, the disinfection or sterilization process involves UV or other forms of disinfecting or sterilizing radiation. The exposed surfaces of the probe in the chamber are subjected to radiation for a predetermined time, after which the radiation is ceased and the probe is removed, ready for use.

Approaches can also be employed in which both chemical and UV sterilization or disinfection are applied sequentially, in any order, or together.

### SUMMARY OF THE INVENTION

Systems and methods in accordance with embodiments of the invention act to retain disinfection/sterilization objects within a disinfection/sterilization chamber in a manner that can reduce occlusion of the disinfection with respect to the disinfecting/sterilizing media.

In an embodiment, the invention provides a device for retaining a medical apparatus in a sterilisation or disinfection apparatus, said device comprising:
a closed cradle configured to fully encircle a portion of said medical apparatus when in use, the cradle having a medical apparatus-facing side comprising a plurality of projections for contact with the medical apparatus, and
a cradle support configured to interface with the sterilisation or disinfection apparatus and retain the cradle within the sterilisation or disinfection apparatus during the operation of the sterilisation or disinfection apparatus.

The medical apparatus may be a probe, for example, an ultrasound probe. More particularly, the apparatus may be wireless, such as a wireless probe or wireless ultrasound probe.

The cradle support may comprise at least one arm extending between an elongate shaft and the cradle. The cradle support may also comprise a spacer intermediate the at least one arm and the elongate shaft. The at least one arm may be offset to one side with respect to a longitudinal axis of the shaft.

In an embodiment, the cradle support comprises two arms extending between the elongate shaft and the cradle. The cradle support may also comprise a spacer intermediate the at least one arm and the elongate shaft. The arms may be offset to one side with respect to a longitudinal axis of the shaft.

In some embodiments, the elongate shaft may comprise a tapered portion for seating in an aperture in the sterilisation or disinfection apparatus. In other embodiments, the shaft does not include a taper (e.g., a taper may not be required for the accessory to be retained within the disinfection chamber).

One or more ribs may be located on the arm or arms and or on the spacer. The ribs may provide mechanical support and/or orient the device within the disinfection or sterilisation apparatus.

The device may include a flattened portion and/or a loop distal to the at least one arm.

The device may be in unitary (i.e. one-piece construction) form. In some embodiments, the device may be composed of a plurality of components. For example, the cradle may define one component and the protrusion(s) may define respective other components.

The device may be formed from any suitable material(s). For example, suitable materials include those materials that are resilient against a utilized disinfectant and/or those materials that can facilitate the delivery of the disinfectant to mated surfaces (e.g. the mating being defined by interface between the medical and the protrusion(s)). Contemplated disinfectants may include hydrogen peroxide and/or peracetic acid. Exemplary materials include nylon, Polyphenylsulfone (PPSU), polycarbonate, PET, polyester, polypropylene, polybutylene terephthalate (PBT), polyvinylidene difluoride (PVDF), or mixtures thereof.

The device may be formed from a material which is UV-transparent. Exemplary materials may include: quartz glass and/or transparent/translucent methacrylate-based polymers.

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment but may. Furthermore, the particular features, structures or characteristics may be combined in any suitable manner, as would be apparent to one of ordinary skill in the art from this disclosure, in one or more embodiments.

### BRIEF DESCRIPTION OF THE DRAWINGS

Embodiments of the invention will now be described, by way of example only, with reference to the accompanying drawings:

FIG. 1 is an embodiment of a device of the present invention.

FIG. 2 shows the engagement of a device in accordance with one embodiment of the present invention with a sterilizing or disinfecting apparatus.

FIG. 3 shows the spacer and arm portion in accordance with one embodiment of a device of the present invention in expanded detail.

FIG. 4 provides examples of the use of the flattened upper portion and loop in accordance with one embodiment of a device of the present invention.

FIGS. 5A and 5B illustrate respectively methods of loading and unloading the device in accordance with one embodiment of the present invention with a probe.

### DESCRIPTION

In recent times cordless probes are emerging as a desirable alterative to corded probes. These have become more common as wireless connectivity and battery technology have improved.

Cordless probes are often preferred by users in a clinical setting, as they are less restrictive in terms of where they can be used, and they are easier to manipulate during patient examination and generally more convenient to handle and store.

The move to cordless probes does however give rise to a new challenge. In addition to providing power and transmitting data from the probe, these cords have been used as de facto supports during storage and cleaning of the probe, enabling the probe to be maintained such a manner that it was not in contact with a surface, which is desirable when the probe is contaminated and essential when the probe is clean. The loss of the cord means that new means of support have to be found.

Cordless instruments are not traditionally able to be suspended in a disinfection or sterilisation chamber due to the prior expectation that any contact points with the probe surface would inhibit disinfection or sterilisation. However prior studies have established that where the contact points are controlled for material and size, a suitable validated level of disinfection or sterilisation can be achieved.

Previous approaches for suspending cordless probes have included net or mesh bags, which can accommodate devices of various configurations. In some circumstances however, alternative means of suspending cordless probes may be preferred.

Exemplary embodiments of the present invention will now be described in detail with reference to the accompanying drawings. In the drawings, the same elements are denoted by the same reference numerals throughout. In the following description, detailed descriptions of known functions and configurations incorporated herein have been omitted for conciseness and clarity.

Ultrasound probes are used for a variety of intra cavity procedures including intra rectal, intra vaginal and oesophageal examination as well as surface use such as prenatal and other inspections. These probes are typically constructed integrally with the power/data cord for powering the probe and linking to a control console. Whilst the probes do not need to be completely sterile in most cases, they do need to be subjected to regular disinfection. Usually, this takes the form of at least a high-level disinfection between each use to prevent cross-contamination. In this regard, the level of contaminant removal achieved by disinfection processes are typically regulated by relevant medical standards to minimise the possibility of the cross-contamination.

Typically, in use, the door of the sterilizing or disinfecting apparatus is opened to expose an internal chamber and the probe for sterilisation or disinfection is then placed inside the chamber and secured in position. The door is then closed, and the sterilisation or disinfection process is commenced.

In one embodiment, a sterilisation or disinfection media in the form of misted/vaporised sterilisation or disinfection fluid is introduced into the sterilisation or disinfection chamber. The fluid is dispersed to swirl around the ultrasound probe during the sterilisation or disinfection process for a predetermined time to assure a desired level of sterilisation or disinfection. This fluid is then dispersed from the chamber (and catalytically destroyed on exit) which may be followed by a flow of air to dry the probe's outer surfaces. At the completion of this process, the outer surfaces of the probe are considered sterilisation or disinfection and can be removed from the probe and used.

The misted sterilisation or disinfection fluid may include a quantity of controlled ultrafine hydrogen peroxide (H₂O₂) or peroxy acetic acid, which enters the chamber through side ports and gently swirls around to cover the entire surface of the probe. The mist particles can penetrate exposed areas of the probe including shadowed areas formed by crevices, grooves and imperfections on the probe surface. As part of the sterilisation or disinfection process, the mist flow is stopped, and a flow of air is introduced to the inside of the chamber so that the exposed surfaces of the probe can dry to a sufficient level. In this regard, it should be understood, that the drying of the probe surfaces can be an important aspect of the sterilisation or disinfection process when using a misted sterilisation or disinfection fluid so that the sterilisation or disinfection fluid can be substantially removed from the probe surfaces at the completion of the process.

The probe is normally secured within the chamber of the sterilisation or disinfection apparatus by way of a clamp disposed at the top of the chamber. This clamp engages with the probe's power/data cord thereby allowing it to be suspended within the chamber during the sterilisation or disinfection process. However, as mentioned above, a problem now exists whereby ultrasound probes, and indeed other medical objects requiring sterilisation or disinfection have been developed with internal power sources and wireless communication devices such that they do not require an external cord. It should be appreciated that in the context of suspending the probe within chamber, the absence of a cord can be problematic in that simply resting the wireless probe within the chamber can inhibit the disinfection media reaching those areas that are in contact with the chamber wall. Resting the wireless probe on the chamber wall may also damage the probes depending on the temperature or not allow for disinfectant to be removed by the drying stage.

Similarly, suspending the probe within the chamber using a conventional bracket, may result in the areas of the probe in contact with the bracket not being sufficiently sterilised or disinfected due to the bracket effectively blocking the sterilisation or disinfection media. In other words, such supportive structures may hinder the sterilisation or disinfection media from reaching respective occluded surfaces. Under these circumstances, the level of contaminant removal may be insufficient to meet regulated medical standards.

With this problem in mind, devices in accordance with embodiments of the invention act to retain a target of sterilisation or disinfection within a sterilisation or disinfection chamber while reducing instances of occlusion and mitigating detrimental impacts of the occlusion—where it does occur—that may otherwise result from corresponding supportive structures.

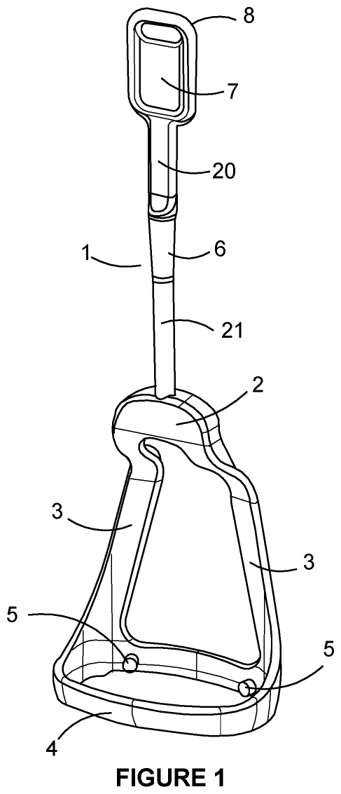

FIG. 1 illustrates one embodiment of a device for retaining an object in a sterilization/disinfection chamber in accordance with certain embodiments of the invention. The illustrated embodiment comprises an elongate shaft 1 attached to a spacer 2, to which are connected two arms 3 which extend downwardly and outwardly from the spacer 2. The arms 3 support a cradle 4 which is in the form of a closed loop, configured to be fully circumferential in use around a medical device such as a probe. An inwardly facing surface of the loop has a plurality of domes or projections 5 for contact with a probe or other medical device.

In this application, the terms “circumferential”, “encircle” may be understood to mean e.g. that the cradle fully surrounds the probe; these terms do not necessarily limit the cradle to a circular shape. The cradle can be for instance a rectangular cradle, hexagonal cradle, square cradle, elliptical, rhomboid shaped cradle etc.

The shaft 1 includes a tapered region 6, where the shaft tapers inwardly towards the spacer. This tapered portion serves to provide a friction or press fit in an aperture of a medical device when the probe holder is in use. The device may be retained by contact at any part of the shaft. In one embodiment, the shaft is held by a clamp or cam. In another embodiment the probe holder is forced downwards under the influence of gravity, pressing the tapered portion into engagement with an aperture normally used to allow the passage or retention of a probe power cord or similar. The aperture is generally of a circular cross section, as are the shaft and tapered region, although it will be appreciated that the probe holder can be of any suitable cross section to correspond with an aperture of the sterilisation or disinfection device. The portion of the shaft 20 above the tapered portion 6 can be generally of equal or larger diameter than the apertures of the sterilization/disinfection device, whereas the portion of the shaft 21 below the tapered portion (i.e. between the tapered portion 6 and the spacer 2) can generally be of equal or smaller diameter than the apertures of the sterilization/disinfection device.

It should be appreciated that while FIG. 1 illustrates one particular form factor for a device for retaining an object in a sterilization/disinfection device, the proposed devices can be of any suitable form factor that can support a closed loop cradle in accordance with embodiments of the invention. For example, in some embodiments, only one arm is used to support the cradle. In some embodiments, the protrusions may be pyramidally shaped.

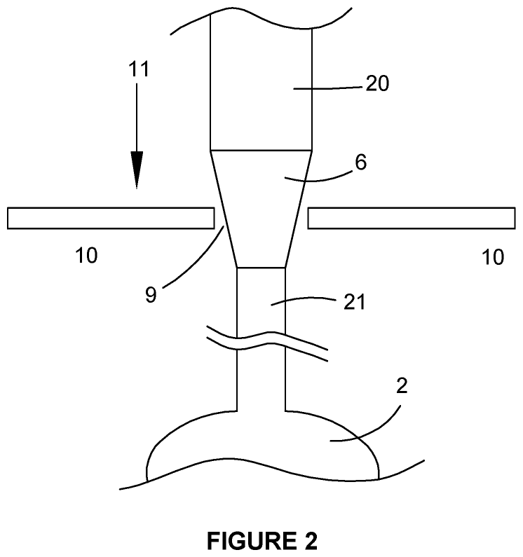

FIG. 2 illustrates how the embodiment of FIG. 1 may be installed within a disinfection/sterilization device in accordance with embodiments of the invention. In FIG. 2, the tapered portion 6 forms a press fit with an aperture 9 in a sterilisation or disinfection device 10 which is normally used to accommodate a cord. In this case, the tapered portion 6 can be seen moving in the direction of the arrow 11 (generally a downward direction) to cause the tapered portion 6 to engage circumferentially with the rim of aperture 9.

In some embodiments of the invention, the weight of a probe in the device may help retain sealing engagement between the tapered portion 6 and the rim of aperture 9.

The tapered shaft 6 allows for compatibility with a number of different clamping mechanisms (such as different spring-loaded clamps and different sized cord apertures).

Again, it should be appreciated that although FIG. 2 illustrates one way for the disclosed devices to interface with a sterilization/disinfection chamber, devices for retaining objects in sterilization/disinfection chambers can interface with sterilization/disinfection chambers in any of a variety of ways in accordance with various embodiments of the invention. For example, an attachment mechanism may be integrated into the shaft in accordance with certain embodiments.

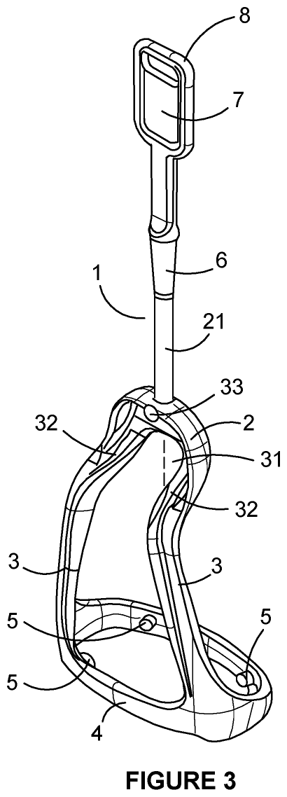

FIG. 3 shows the reverse side of the embodiment of FIG. 1.

In FIG. 3 it can be seen that the spacer 2 supports the two arms 3 in an arrangement such that the arms extend outwardly from the spacer and further that both arms are cantilevered to be offset one side of the axis 31 defined by the shaft. This arrangement means that when a probe is located in the cradle, the arms are located towards the corners of the probe on one side.

This arrangement is such that the centre of gravity of the probe is maintained at or very near to the vertical axis of the shaft 31. Further, probe is supported within an area defined by the arms 3. In this manner, the device can provide stability of the probe when retained and can further serve to minimises fatigue and stress on the holder, where bending forces at various points of the holder are obviated or minimised and only tensile forces predominate.

Devices in accordance with various embodiments of the invention may include strengthening formation, such as ribs, at various locations if required. For example, the arms may further include one or more ribs. These can provide mechanical support for the cantilevered arms at or near bends or where there are changes in cross section, such as for instance rib 32 where the arm is attached to the spacer. Also encompassed by the present invention are embodiments which implement more than two (2) arms, for example, the device may include three (3) arms or four (4) arms. Each arm may be the same or they may be of different configuration, for example, to accommodate alternative chamber configurations.

Further, the device may include one or more ribs or one or more other projections as a means to assist in locating or orienting the holder in a steriliser in a correct or preferred location. In one embodiment, the device includes a projection or rib 33 positioned on the spacer portion to prevent false location in a steriliser.

The arms 3 can be of any length, however, they are sized to be of sufficient length to accommodate a wireless probe in the cradle, while allowing a sterilisation or disinfection fluid to contact the device freely and without the arms causing occlusion or shadowing of the device from sterilant. The arms also need to be sized to fit within a sterilisation or disinfection chamber.

The cradle 4 is fully circumferential with respect to the probe designed to be held and sized appropriately. The cradle is sized to provide sufficient clearance all around the probe and to ensure good sterilisation or disinfection and avoid potential shadowing of areas.

In many embodiments, the cradle itself does not directly contact the probe, but rather, the inner surface of the cradle comprises a plurality of projections 5 which are positioned to contact the probe and to maintain the probe in a spaced apart arrangement with the cradle 4 and/or other parts of the device, such as the arms 3 or spacer 2. That is, in use, the only point of contact between the probe and the device is the projections 5 on the inner surface of the cradle.

The projections may be generally configured so as to minimise the extent of occlusion of the surface probe surface by the probe holder.

The projections 5 may thus be formed so as to be in the form of a cone, dome or similar, with the smallest practicable surface area in contact with the probe, so as to ensure a minimum of mated surface contact with the probe, thereby to allow maximum contact with the sterilisation or disinfection fluid.

In one embodiment, four projections 5 are provided at the inner surface of the cradle 4. Any number of projections may be used, bearing in mind the need for stability of the probe on the one hand and need for a minimal total surface area of the probe being contacted with the projections.

The height of the projections 5 is such that the device is also distanced from the cradle 4 to avoid shadowing and allow free contact between the probe surface and sterilisation or disinfection fluid.

In some embodiments, protrusion(s) may also be on parts of the device other than the inner surface of the cradle. For example, the protrusions may be present on the outer surface of the cradle, on one or more of the arms, on the spacer or on the shaft. The protrusions may serve, for example, to aid in alignment of the holder in the sterilising apparatus or to minimise contact interference between the holder and other object.

The protrusions may be formed integrally with the holder, or they may be removable. For example, the protrusions may be separately formed consumables, with the protrusion and holder having complementary detent means to retain the protrusion in the contact with the holder. In one such embodiment, the protrusion is in the form of a cap that plugs into (or screws into or otherwise engages) a socket on the holder. In an alternate embodiment, the protrusion is in the form of a cap that fits onto a stub (for example by press fit, screw fit or other means of engagement). The protrusions can then be replaced between sterilisation cycles or as they wear out.

Separately formed protrusions may be provided in a variety of shapes and sizes, which enables the selection of an appropriately configured protrusion to match a variety of probe types, for instance, a larger protrusion on the interior surface may be selected when a smaller probe is to be accommodated. The ability to mix and match protrusions can enable a wide range of probe configurations to be retained for sterilisation.

The protrusions (either those integrally formed with the cradle or consumable) may be configured to accommodate adaptors which can fit over the protrusions, for example, a cap that can be put on to the protrusion such that it protrudes further. Such caps may for example be retained over the protrusion by a simple press fit and if desired removed after use.

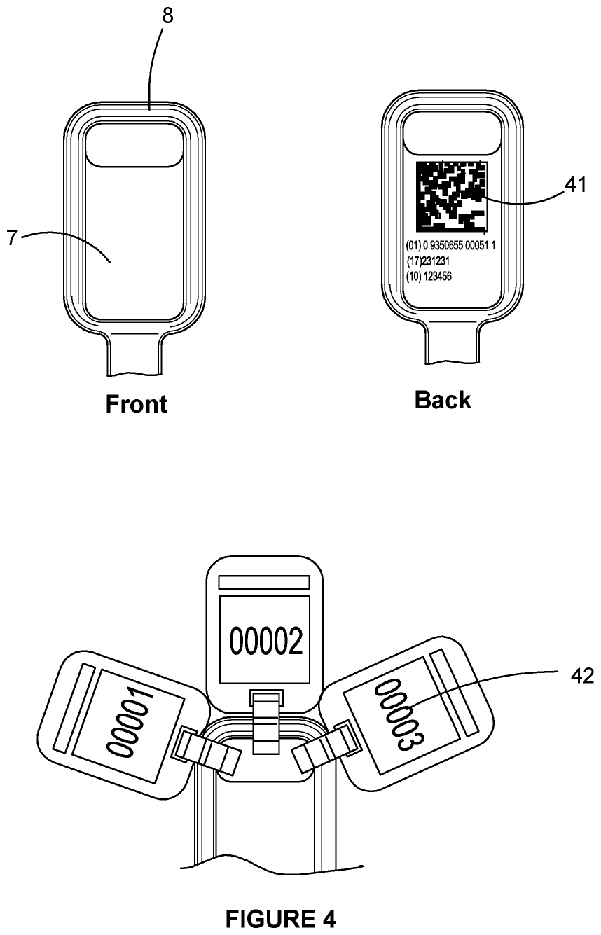

The holder may also have an upper portion to facilitate handling and/or identification of the holder and/or of probes retained therein. With reference to the drawing, FIG. 4 shows flattened portion 7 located towards the top of the device and above the tapered portion. Flattened portion 7 projects from the steriliser when in use and serves to enable the holder to be gripped or handled without contacting a contaminated probe (prior to disinfection) or a sterile probe (after disinfection) which is located in the cradle below the tapered portion.

As well as providing a point of manual contact, the flattened portion may also serve as useful for bearing indicia 41 and the like, for example, barcodes, QR codes, RFID chips and so on which can be used to track the holder and/or the probe.

Additionally, the device may also comprise a loop 8 also located above the sterile portion which can be used to assist in storage of the device such as to be hung from a hook, as well as to allow for the attachment of tags 42 or other identifiers. These identifiers can be used to track the holder and/or the probe and provide real time information as to the status and location of probes and or probe holders. Again, the tags can be for example, barcodes, QR codes, RFID chips and so on.

In several embodiments, the device allows for attachment of a unique identifier, to enable traceability of the device or probe to the particular sterilisation or disinfection cycle. The unique identifier may be in the form of a digital solution such as a RFID tag or barcode/QR code. Alternatively, the unique identifier may be an analogue solution such as a serial number. In another embodiment, the supporting member shall have an embedded unique identifier, to enable traceability of the supporting member to the particular sterilisation or disinfection cycle.

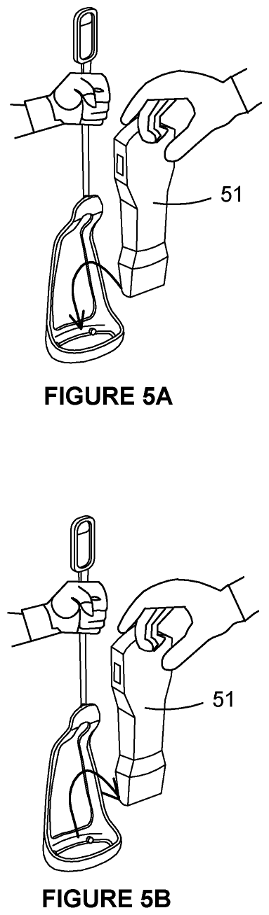

The disclosed form factors can facilitate ease of use of the retaining mechanisms. FIG. 5 illustrates how probes may be inserted with the disclosed retainers in accordance with embodiments of the invention. In one embodiment, the probe 51 is first loaded into the cradle 4, to interface with the protrusions 5. The ‘top-loading’ nature procedure allows for simplicity and largely a foolproof operation. For example, gravity can force the probe to interact with the protrusions in the desired placement/orientation on a consistent basis. It should be appreciated that the ease of loading can also be applied to the ease of unloading. With the unloading process being the reverse process, with lifting the probe out of the cradle 4.

When the probe is loaded onto the protrusions 5 the weight of the probe is supported by the shaft 1 and the assembly can be subsequently inserted into the sterilizer where only the shaft 1 is supported, where it is retained for sterilisation or disinfection.

Alternatively, the retaining mechanism may be placed in the sterilizer first and then subsequently loaded with the probe.

After sterilisation or disinfection is complete, the retaining mechanism invention may be removed together from the sterilizer, or the probe may be removed from the retaining mechanism.

The disclosed retaining mechanisms can be of unitary construction, in order to avoid the creation of joints and mated surfaces which may compromise sterilisation or disinfection. In some embodiments, the device may be composed of a plurality of components. For example, the cradle may define one component and the protrusion(s) may define other respective components.

The device may be moulded, e.g. ejection moulded, printed or formed via shaping.

The surface of the device may be polished to remove potential sites which can harbour microorganisms, or the device may have a coating of a suitable material applied to ensure a smooth surface, and/or an antibacterial surface and/or a surface which has enhanced resistance to a sterilisation or disinfection agent.

The device may be formed from any suitable material(s). For example, suitable materials include those materials that are resilient against a utilized disinfectant and/or those materials that can facilitate the delivery of the disinfectant to mated surfaces (e.g. the mating being defined by interface between the medical and the protrusion(s)). Contemplated disinfectants may include hydrogen peroxide and/or peracetic acid. Exemplary materials include nylon, polyphenylsulfone (PPSU), polycarbonate, PET, polyester, polypropylene, polybutylene terephthalate (PBT), polyvinylidene difluoride (PVDF), or mixtures thereof. These materials may have favourable wicking properties that can help liquid chemical disinfectant better penetrate mated surfaces. As can be appreciated, different components can be formed from different materials.

Some disinfection/sterilization apparatus utilize UV (ultraviolet) or Gamma radiation as disinfection media, which may be emitted from internally mounted lamp(s) or other source(s), to reach the external surfaces of the object undergoing disinfection. UV or Gamma radiation has a strong bactericidal effect. It can be absorbed by the DNA of any microorganism that is present, to destroy its structure and inactivate any living cells. Microorganisms such as viruses, bacteria, yeasts and fungi may be rendered harmless within seconds by UV radiation.

Thus, the disclosed retaining mechanisms may be made from material that is transparent to UV-radiation and/or gamma radiation in accordance with various embodiments of the invention. For example, suitable materials may include: quartz glass and/or transparent/translucent methacrylate-based polymers. Of course, it should be appreciated that any suitable material may be used in accordance with embodiments of the invention.

In this way, accessories for use within sterilisation or disinfection chambers that are based on the application of electromagnetic radiation can be achieved.

The disclosed retaining mechanisms, when made from radiation transparent material, may be particularly suited to mixed mode disinfection where both a sterilizing fluid and electromagnetic radiation are used, for instance, combination UV/peroxide or UV/peracetic acid sterilizing systems. For example, by utilising a material which is both effectively transparent to UV-radiation and effectively transparent to a disinfecting chemistry. Alternatively, the main body of the mechanism may be made from UV-transparent materials, while the protruding component(s) of the mechanism may be made of materials suitable for facilitating the delivery of disinfectant chemistry to a corresponding mated surface. In this way, (1) disinfecting chemistry may reach the entirety of the object to be disinfected, and (2) disinfecting radiation (e.g. UV and/or Gamma-radiation) can reach the majority of the object to be disinfected (the radiation may be occluded by the protrusions in this example).

The above-described examples can be augmented and enhanced in various ways in accordance with embodiments of the invention.

In some embodiments, the device of the present invention may be a single or limited use item. Under these circumstances, at least a portion of the support member may be impregnated with a dye that reacts to the sterilisation or disinfection media so that the support member will change color at the completion of a given number of sterilisation or disinfection processes. In this way, a visual indication is provided to the user to easily determine if the device is suitable for use.

It should be appreciated that in the above description of exemplary embodiments of the invention, various features of the invention are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims are hereby expressly incorporated into this detailed description, with each claim standing on its own as a separate embodiment of this invention.

In the description provided herein, numerous specific details are set forth. However, it is understood that embodiments of the invention may be practiced without these specific details.

In other instances, well-known methods, structures and techniques have not been shown in detail in order not to obscure an understanding of this description.

Those skilled in the art will recognize that other and further modifications may be made thereto without departing from the spirit of the invention, and it is intended to claim all such changes and modifications as falling within the scope of the invention. Steps may be added or deleted to methods described within the scope of the present invention.

1. A device for retaining a medical apparatus in a sterilization or disinfection apparatus, said device comprising:
a closed cradle configured to encircle a portion of said medical apparatus when in use, the cradle having a medical apparatus-facing side comprising a plurality of projections for contact with the medical apparatus, and
a cradle support configured to interface with the sterilization or disinfection apparatus and retain the cradle within the sterilization or disinfection apparatus during the operation of the sterilization or disinfection apparatus.

2. The device according to claim 1 wherein the cradle support comprises at least one arm extending between an elongate shaft and the closed cradle.

3. The device according to claim 2 wherein the cradle support comprises a spacer intermediate the at least one arm and the elongate shaft.

4. The device according to claim 2 wherein the at least one arm is offset to one side with respect to a longitudinal axis of the shaft.

5. The device according to claim 2 wherein the at least one arm comprises two arms extending between the elongate shaft and the closed cradle.

6. (canceled)

7. The device according to claim 2 wherein the at least one arm is offset to one side with respect to a longitudinal axis of the shaft.

8. The device according to claim 2 wherein the elongate shaft comprises a tapered portion for seating in an aperture in the sterilization or disinfection apparatus.

9. The device according to claim 2 comprising one or more ribs located on the at least one arm.

10. The device according to claim 3 comprising one or more ribs on the spacer.

11. The device according to claim 9 wherein the ribs provide mechanical support.

12. The device according to claim 9 wherein the ribs orient the device within the sterilization or disinfection apparatus.

13. The device according to claim 2, further including a flattened portion distal to the at least one arm.

14. The device according to claim 2, further including a loop distal to the at least one arm.

15. (canceled)

16. The device according to claim 1, wherein at least one of the plurality of protrusions are removable.

17. The device according to claim 16 wherein the at least one of the plurality of protrusions is in the form of cap that engages a socket in the closed cradle.

18. The device according to claim 16 wherein the at least one of the plurality of protrusions is in the form of cap that engages a stub on the closed cradle.

19. The device according to claim 1, wherein the closed cradle and the cradle support are formed from a material resistant to a predetermined disinfectant or sterilant.

20. The device according to claim 1, wherein the closed cradle and the cradle support are formed from nylon, polyphenylsulfone (PPSU), polycarbonate, PET, polyester, polypropylene, polybutylene terephthalate (PBT), polyvinylidene difluoride (PVDF), or mixtures thereof.

21. The device according to claim 1, wherein the closed cradle and the cradle support are formed from a material which is UV-transparent.
